(19) United States
(12) Patent Application Publication
(10) Pub. No.: US 2005/0177155 A1
(43) Pub. Date: Aug. 11, 2005

(54) Anterior adhesion resistant barrier for spine

(76) Inventor: Neville Alleyne, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/976,175

(22) Filed: Oct. 28, 2004

Related U.S. Application Data
(60) Provisional application No. 60/515,084, filed on Oct. 28, 2003.

(51) Int. Cl. A61B 17/56

(52) U.S. Cl. 606/61

(57) ABSTRACT

A shield is configured for implantation over an anterior portion of a spinal column is provided. The shield comprises a sheet of material having first and second adjacent portions, wherein the first portion defines a first shield width, and wherein the second portion defines a second shield width that is wider than the first shield width. Furthermore, a method of protecting a surgical site on an anterior portion of a spine comprises placing a shield over a spinal surgical site on an anterior spinal location between at least one spinal disk space and adjacent blood vessels.

### CROSS RELATED APPLICATIONS

This application claims priority to U.S. Provisional Application No. 60/515,084, filed on Oct. 28, 2003, which is incorporated herein by reference in its entirety.

### BACKGROUND OF THE INVENTION

Anterior spinal surgery has many advantages and some disadvantages. In the last decade we have seen the emergence of minimally invasive surgery being done through small mini anterior incisions, gaining access via the retroperitoneal dissection down to the anterior vertebral column. We have also seen anterior midline incisions and transperitoneal incisions to the lumbar spine. These anterior approaches can be anterior retroperitoneal flank approaches or anterior extraperitoneal midline incisions or transperitoneal midline approaches. With these aforementioned approaches as part of the armamentarium for the orthopaedic and spine surgeon anterior column stability can be achieved since we know between 75 and 80% of the support occurs through the anterior column. Given the ability to have access to the anterior column has allowed surgeons in the last two decades to achieve an arthrodesis with allograft bone, threaded fusion cages-metallic and nonmetallic, impaction cages, as well as plate fixation. Achieving solid arthrodesis is essential in spinal surgery. However, migration of these devices secondary to infection or mere loosening has resulted in disasters in some cases. These disasters have included vascular injury and significant bony destruction to the vertebral body in order to retrieve the implant. Over the last two decades prior anterior surgery has been a relative contraindication unless infection or migration of the implants cannot be controlled with a more traditional posterior approach or posterolateral approach. It has therefore been a significant problem for access surgeons to mobilize the great vessels at or cephalad to L4-5. This is partly due to fibrosis that occurs in the retroperitoneal space and causes adherence to the aorta, vena cava and the other retroperitoneal structures referred to in the paragraphs below.

### SUMMARY OF THE INVENTION

In one embodiment, a shield configured for implantation over an anterior portion of a spinal column is provided. The shield comprises a sheet of material having first and second adjacent portions, wherein the first portion defines a first shield width, and wherein the second portion defines a second shield width that is wider than the first shield width.

In addition, a method of protecting a surgical site on an anterior portion of a spine is provided. The method comprises placing a shield over a spinal surgical site on an anterior spinal location between at least one spinal disk space and adjacent blood vessels.

### BRIEF DESCRIPTION OF THE DRAWINGS

FIGS. 1A-1D illustrate several shield embodiments in accordance with some aspects of the invention;

FIG. 2 illustrates an advantageous area of shield position following anterior spinal surgery.

### DETAILED DESCRIPTION OF THE PREFERRED EMBODIMENT

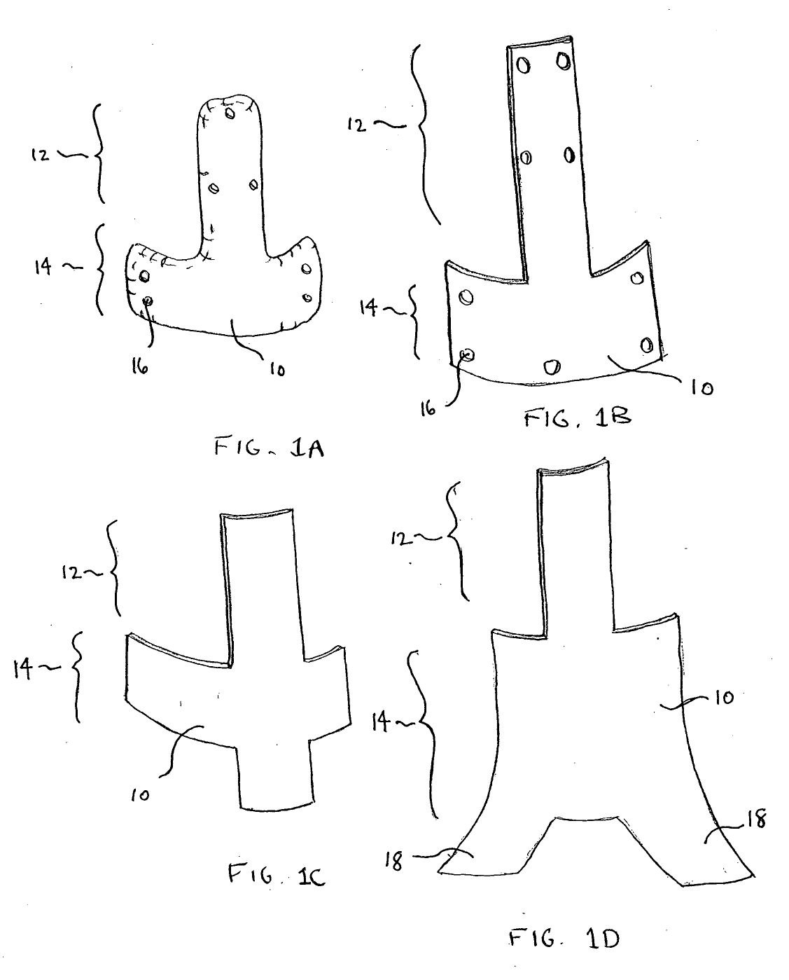

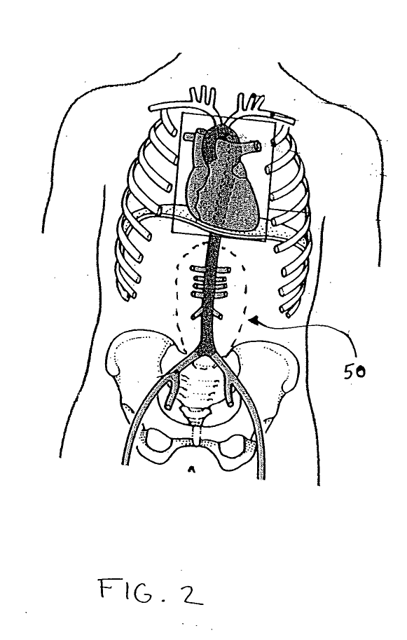

Several embodiments of a shield or barrier according to the invention are illustrated in FIGS. 1A through 1D. The device may comprise a plastic material which can be applied over an annular defect to minimize the amount of scar tissue and facilitate revision surgery. As shown in FIGS. 1A-1D, the shield 10 may be formed with a first portion 12 having a first width, and a second portion 14 having a second width wider than the first width. Thus, the device may advantageously be T or cross-shaped. Such a device would be anchored typically on the anterior portion of the spinal column into the end plate above and below in order to cover the annulotomy defect and/or disk space implant. The general location of installation for anterior lumbar and thoracic applications is shown in FIG. 2, designated 50, beneath the aorta and vena cava. This material would not insinuate itself into the disc space to impede the natural 6° of freedom in the motion segment. Such a device would minimize contact between the spinal surgery site and the great vessels and/or ureter or retroperitoneal tissue and structures. These would include, but are not limited to the aorta, vena cava, common iliac artery, veins, lymph nodes, and cistema chyli. The lumbosacral plexus or trunk, including the gray rami communicantes, the muscles, such as the iliacus, the psoas major and minor, the quadratus lumborum, branches of the diaphragm, the tranversis abdominis, thoracic duct, the ureter, anterior longitudinal ligament and the brim of the pelvis. As a result the borders of this retroperitoneal space anteriorly is the peritoneum posteriorly by the spine, psoas, quadratus lumborum muscles, superiorly by the twelfth rib and attachments of the diaphragm, and lastly inferiorly by the brim of the pelvis. The lateral margins of the retroperitoneal space corresponds to the lateral borders of the quadratus lumborum muscles. These both the actual and potential space of the solid organs and major blood vessels. The solid structures, including the kidney, ureters, adrenal glands, portions of the autonomic and peripheral nervous system, pancreas, abdominal aorta, inferior vena cava, spermatic or ovarian vessels, lymphatics and lymph nodes and certain portions of the intestinal tract, notably the duodenum occupies this space, along with fatty areolar tissue and fibrous connective tissue.

The device may be shaped to mimic the convex border and contour of the disc anteriorly with anchors that would then go a few millimeters into the vertebral body and/or end plate above and below. The device may have thicknesses varying from about 0.1 up to about 10 mm, preferably about 0.25 to about 0.5 mm and with anchors that would go into the anterior vertebral body or end plate 5-10 mm. These anchors can be an integral part of the anterior barrier or they can be engaged after the device is applied over the involved annulotomy. FIGS. 1A and 1B illustrate holes 16 for anchor or suture placement. FIGS. 1C and 1D illustrate embodiments with no openings. These may be placed with no anchoring at all if desired. When used, the anchoring teeth can be made of the same material or can be made of metal, plastic, methacrylate, ceramic, titanium, molybdenum, stainless steel, tantalum, etc. The teeth serve only to anchor the barrier and prevent migration. No structural support or fixation is desired. The surfaces will be smooth, and there will preferably be no sharp edges to compromise the vascular structures in this region. In some embodiments, as shown in FIG. 1A, one or more corners and/or edges of the device may be rounded to truly eliminate all possible sources of tissue abrasion. This is important near the great vessels as they expand and contract with the blood pressure and it is important to avoid the possibility that repetitive motion against the barrier will damage these vessel walls.

Since the geometry of this device mimics the anterior portion of the disc space and end plates that may be made in variable heights (compare, for example, FIGS. 1A and 1B), the entire annulotomy and surgical working region at the disc space should be covered. For level at L4-5 (FIG. 1C is one possible L4-5 embodiment) and cephalad segments placing a very thin piece of this material and anchoring it down into the vertebral body or end plates can be done after the annulotomy region has been addressed. As shown in FIG. 1D, the lower portion of the barrier can be configured with branches 18 to follow the vessels after branching at the base of the spinal column. If used anteriorly the device can be extended above L4-5 to lay on the surface between the great vessels and the anterior longitudinal ligament from L1-2 down through L4-5. At L5-S1 since the great vessels are not anterior, the device can be implanted at L5-S1 with anchors to the vertebral body at L5 and the sacrum below.

This non-biodegradable material can be made of Carbothane (a polyurethane material) or any other assorted materials well known to surgeons in this field. In these embodiments, the device is not bio-absorbable but is preferably a pliable material with a relatively low flex modulus such that it is easily bendable by hand. The barrier may also have a radiopaque material to identify its position as well as the anchors. This will facilitate the surgeons at a later date to compare with postoperative AP and lateral x-ray, whether or not the implant has migrated especially if one is contemplating repeat anterior surgery. In addition, for high risk patients with severe osteoporosis or who are smokers, the periphery of the barrier may have impregnated a bone stimulator in addition, in the region of the disc space.

It is also possible to utilize biodegradable implants to cover the annulotomy defect, as well as cephalad segments. The material may be made of PLA, PGA, PLLA, or any assorted variety which is well known to surgeons in this field. A bioabsorbable barrier accommodates those access surgeons or orthopaedic or spine surgeons that feel a non-biodegradable may increase the potential risk for infection and the advantages of the bioabsorbable is that after several months the material will be gone, and the ease for reentering that level disc space should still be greatly enhanced. The proximal mobilization of the great vessels, vena cava, aorta with its tributaries and branches respectively can also be easily accessed. With the bioabsorbable barrier, anchors can be either bioabsorbable or non-bioabsorbable and will still be compatible with the bioabsorbable implant. The device may come in absorptions that would allow the device to degrade at six months or one year. As a separate attachment the device can have a micro pump to deliver a precise amount of the anti-adhesion fluid, such as mineral oil to this region to inhibit retroperitoneal adhesions. This pump would have a finite amount of anti-adhesion fluid that would lubricate the surface of this implant. The use of the mineral oil in the general surgeon and OB-GYN literature has been supported for years, or any additional lubricants, such as Heparin, sulfate derivatives, etc. The device may contain scar retarding substance, such as Heparin sulfate, glycerin (similar clot retarding substances being used in cardiology with regard to stents), etc., and may be prepared in a color that contrasts with the colors of the viscera and skeletal tissue. The device may also be utilized by a nano technology or memchip technology to deliver pain medication, gene therapy or additional nutrients to the disc.

By facilitating reentry into the disc space we also facilitate mobilization of the great vessels which could occur with radiation treatment, tumor, both benign and malignant, metastatic disease, idiopathic retroperitoneal fibrosis or prior retroperitoneal surgery. Any breach in the retroperitoneal space could certainly induce such fibrosis to occur. Therefore, the device is essential in facilitating revision surgery because it will minimize significant morbidity and mortality. With the increased number of anterior surgeries being performed, the number of revision surgeries will increase. A portion of this increase in revision surgery will be due to infection, migration, adjacent segment disease, and improper placement of devices initially. For many of these procedures mentioned anteriorly, posterior salvage surgery may not be the answer. It is the inventor's opinion that failed spinal arthroplasties will most likely need to be removed by an anterior approach with better retrieval instruments versus going posteriorly and placing pedicle screws and performing a posterolateral fusion. It is the inventor's believe that the posterior salvage procedures will fail due to pseudarthrosis and/or facture or loosening of the posterior instrumentation. This will then condemn the patient to additional surgery to have the posterior instrumentation removed and also necessitate a repeat anterior approach being done which may include additional levels.

The invention thus facilitates retrieval of anterior interbody instrumentation used for (ALIF or X-LIF or spinal arthroplasty (artificial disc)). The device can also be used posterolaterally or anterolaterally or posteriorly to facilitate posterior lumbar interbody fusion, or TLIF. As a result, any annular defect in the motion segment can now be covered without impeding the interbody device or arthroplasty or bone graft or nuclear tissue. The device will facilitate surgery at this level by covering the defect and allowing the access surgeon ease in identifying the prior surgical level. For adjacent segments the barrier can have an extension piece onto the anterior vertebral column that lays below the great vessels, (aorta, vena cava, branches and tributaries) to facilitate entry at adjacent segments above or below the operated level.

Embodiments of the invention provide protection of the operated level for reentry and also facilitates cephalad mobilization of the great vessels and their branches and tributaries. With such mobilization being facilitated by a mechanical barrier that is either non-absorbable or bioabsorbable (but most preferable non-absorbable), the access surgeons can minimize the amount of time, bleeding, neurovascular injury and bony destruction to the motion segment involved. At the present time there is no anterior barrier or shield for a disc space after an annulotomy is performed for an ALIF or for an artificial disc or after discectomy. Such a device will facilitate revision surgery of the same disc space or cephalad motion segments to L4-5, therefore addressing segment disease with an interbody fusion, discectomy, artificial disc or plate fixation.

1. A shield configured for implantation over an anterior portion of a spinal column, said shield comprising a sheet of material having first and second adjacent portions, wherein said first portion defines a first shield width, and wherein said second portion defines a second shield width that is wider than said first shield width.

2. The shield of claim 1, wherein said shield comprises a biodegradable material.

3. The shield of claim 1, wherein said shield comprises polyurethane.

4. The shield of claim 1, wherein said shield has a thickness of about 0.1 mm to about 10 mm.

5. The shield of claim 1, additionally comprising anchoring ports.

6. The shield of claim 1, wherein said second portion is configured to follow the anterior portion of the disk space.

7. The shield of claim 1, wherein said second portion comprises branches configured to follow vessels after branching from the base of the spinal column.

8. The shield of claim 1, wherein said shield is impregnated with a radio-opaque material.

9. The shield of claim 1, wherein said shield comprise a pharmaceutical substance to inhibit adhesion formation or enhance wound and/or bone healing.

10. A method of protecting a surgical site on an anterior portion of a spine, said method comprising placing a shield over a spinal surgical site on an anterior spinal location between at least one spinal disk space and adjacent blood vessels.

11. The method of claim 10, wherein said device is positioned between a disk space and the aorta and vena cava.

12. The method of claim 10, additionally comprising anchoring said shield to a vertebral body.
